(19) United States
(12) Patent Application Publication
(10) Pub. No.: US 2013/0037034 A1
(43) Pub. Date: Feb. 14, 2013

(54) TRACHEOTOMY SHIELD

(71) Applicants: Wayne W. Disanza, Toms River, NJ (US); Robert M. Landis, Mountainside, NJ (US)

(72) Inventors: Wayne W. Disanza, Toms River, NJ (US); Robert M. Landis, Mountainside, NJ (US)

(73) Assignee: Airways Development LLC, Kenilworth, NJ (US)

(21) Appl. No.: 13/533,320

(22) Filed: Jun. 26, 2012

Related U.S. Application Data
(60) Provisional application No. 61/522,466, filed on Aug. 11, 2011.

(51) Int. Cl. A61M 16/04

(52) U.S. Cl. 128/207.14

(57) ABSTRACT

A tracheotomy shield includes a first sheet defining an opening extending therethrough. The opening in the tracheotomy shield is configured to receive a portion of a tracheotomy tube assembly and to establish a liquid-tight seal about the portion of the tracheotomy tube assembly. The first sheet is wholly supported by the tracheotomy tube assembly.

### CROSS-REFERENCE TO RELATED APPLICATIONS

This application claims the benefit of, and priority to, U.S. Provisional Patent Application No. 61/522,466, filed on Aug. 11, 2011, the entire contents of which are hereby incorporated by reference herein.

### BACKGROUND

### Technical Field

The present disclosure relates to medical devices and, more particularly, to a protective shield for a tracheotomy tube.

### Description of Related Art

A tracheotomy is a common surgical procedure that involves making an opening, or stoma, through the neck and into the trachea or windpipe of a patient. A tracheotomy tube is then inserted through the stoma into the trachea, allowing the patient to breathe through the tube instead of through the nose and mouth. A tracheotomy may be a temporary measure to support various treatments or to manage an acute episode. In some cases, a tracheotomy is or becomes permanent.

The components of tracheotomy tube assemblies or kits are well known to those skilled in the art. The tracheotomy tube is typically sized to fit the size of the patient's trachea. No matter the size of the tracheotomy tube, the outer most portion of the tube assembly is a 15 mm fitting configured to mate with various breathing systems such as ventilators, resuscitators, and oxygen delivery devices. The tracheotomy tube extends from the 15 mm fitting through the patient's neck and into the trachea. The portion of the tube inside the patient's trachea often has an inflatable cuff that makes a seal in the trachea around the tube. Some tubes have an inner cannula that fits inside the lumen of the tube to facilitate cleaning and maintaining an open airway. Typically, the tube is threaded through a flange distal to the 15 mm fitting and has slots at either end for attaching straps that go around the patient's neck to secure the tube in place. Such configurations are well known to those skilled in tracheotomy care.

A tracheotomy dressing is an absorbent material or pad that is typically positioned about the tracheotomy tube between the neck plate and the patient's skin to protect the tracheotomy wound, e.g., the stoma. Examples of such tracheotomy dressings are described in U.S. Pat. Nos. 3,422,817 to Mishkin et al., 3,585,997 to Ancerewicz, Jr., 5,000,741 to Kalt, 5,058,579 to Terry et al., and 5,918,599 to Shesol. These and other such dressings are widely used and are the standard for tracheotomy care in medical facilities and in the home. These dressings need to be changed once they become soiled with exudate from the tracheotomy wound or from secretions expelled from the airway. Non-absorbent pads, such as that described in U.S. Patent Application Publication No. 2009/0126740 to Loescher, are also known in the art for catching and holding exhaled secretions.

### SUMMARY

In accordance with the present disclosure, a tracheotomy shield is provided including a first sheet defining an opening extending therethrough. The opening defined within the first sheet is configured to receive a portion of a tracheotomy tube assembly and to establish a liquid-tight seal about the portion of the tracheotomy tube assembly. The first sheet is wholly supported by the tracheotomy tube assembly.

In embodiments, the first sheet is formed from a ripstop material.

In embodiments, the first sheet is formed from a transparent material.

In embodiments, the first sheet defines a concave upper edge. The opening may be positioned towards the concave upper edge of the first sheet.

In embodiments, a second sheet is disposed atop the first sheet. The second sheet is attached to the first sheet about at least a portion of outer peripheries of the first and second sheets.

In embodiments, the second sheet defines a generally U-shaped configuration and is attached to the first sheet on three sides.

In embodiments, the second sheet defines a free inner peripheral edge forming a pouch between the first and second sheets.

In embodiments, the second sheet is attached to the first sheet via welding.

In embodiments, the second sheet is formed from a ripstop material.

A tracheotomy system is also provided in accordance with the present disclosure. The tracheotomy system includes a tracheotomy tube configured for positioning within an airway of a patient, a fitting configured for engagement about a free end of the tracheotomy tube, and a tracheotomy shield. The tracheotomy shield includes a first sheet defining an opening extending therethrough. The opening is configured to receive a portion of the fitting and to establish a liquid-tight seal about the fitting. The first sheet is wholly supported by the fitting.

In embodiments, the fitting includes an annular shoulder configured to seat the first sheet thereon. The tracheotomy shield may additionally or otherwise be configured similarly to any of the embodiments above.

Another tracheotomy shield provided in accordance with the present disclosure includes a first sheet defining an opening extending therethrough. The opening is configured to receive a portion of a tracheotomy tube assembly and to establish a liquid-tight seal about the portion of the tracheotomy tube assembly. The first sheet is wholly supported by the tracheotomy tube assembly. A second sheet defines a generally U-shaped configuration and is attached to the first sheet about at least a portion of outer peripheries of the first and second sheets. A pouch is defined between the first and second sheets. The second sheet is configured to provide structural support and balance to the first sheet to maintain proper positioning of the first sheet when supported by the tracheotomy tube assembly.

Another tracheotomy shield provided in accordance with the present disclosure includes a single sheet cut to a length that allows the bottom half to be folded up over the top half to form a loop or sling. In this embodiment, the sides are then sealed to create a pouch area. The pouch areas include the lateral and lower portions of the shield where secretions are contained.

Another tracheotomy shield provided in accordance with the present disclosure includes a single sheet made of a material that is transparent to allow visualization of the tracheotomy assemblies below the shield and cut to a length that allows the bottom half to be folded up over the top half to form a loop or sling. In this embodiment, the sides are then sealed to create a pouch area. The pouch areas include the lateral and lower portions of the shield where secretions are contained.

Any of the tracheotomy shields described above may include any or all of the features of any of the other tracheotomy shields described above.

### BRIEF DESCRIPTION OF THE DRAWINGS

Various embodiments of the present disclosure are described herein with reference to the drawings wherein:

FIG. 1 is a front, perspective view of a tracheotomy shield provided in accordance with the present disclosure;

FIG. 2 is a front view of the tracheotomy shield of FIG. 1;

FIG. 3 is a rear view of the tracheotomy shield of FIG. 1;

FIG. 4 is a front view of another tracheotomy shield provided in accordance with the present disclosure;

FIG. 5 is a front view of the tracheotomy shield of FIG. 1 supported on a tracheotomy tube of a patient;

FIG. 6 is a cross-sectional view of the tracheotomy shield of FIG. 1 supported on a tracheotomy tube of a patient;

FIG. 7 is a front view of a single sheet of material used to form a tracheotomy shield in accordance with the present disclosure;

FIG. 8 is a front view of the single sheet of material of FIG. 7 with a fold line indicated;

FIG. 9 is perspective view of the single sheet of material of FIG. 7 having been folded about the fold line of FIG. 8 to create a loop or sling; and

FIG. 10 is a front view of the single sheet of material of FIG. 7 wherein the sides have bee joined to one another to form the tracheotomy shield with a pouch area on the sides and base thereof.

### DETAILED DESCRIPTION

Embodiments of the present disclosure are described in detail with reference to the drawing figures wherein like reference numerals identify similar or identical elements.

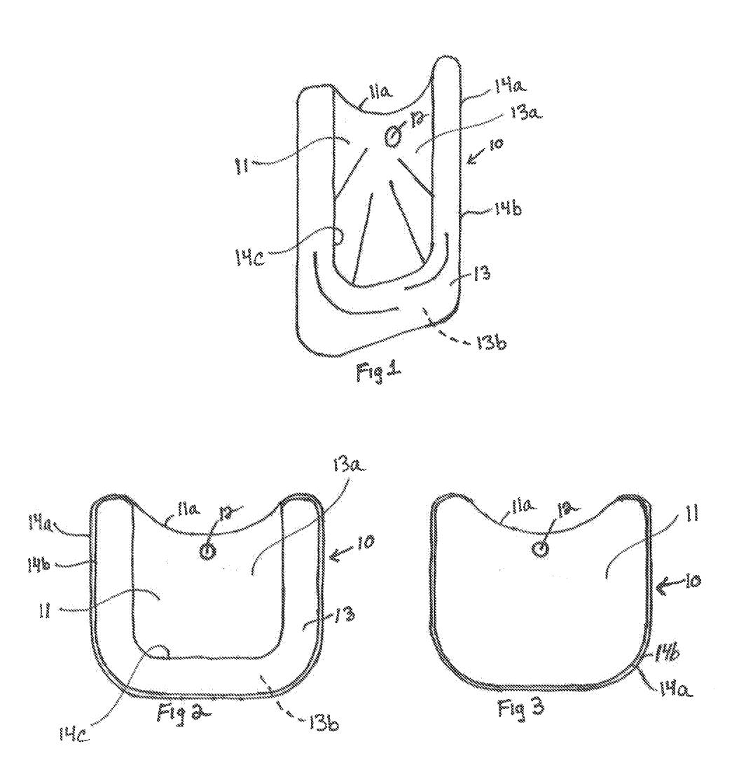

Referring to FIGS. 1-3 and 5, a tracheotomy shield provided in accordance with the present disclosure is shown generally identified by reference numeral 10. Tracheotomy shield 10 generally includes a back sheet 11 defining an opening 12 therethrough, and a front sheet 13 attached to back sheet 11 along at least a portion of the outer peripheries 14a, 14b of back and front sheets 11, 13, respectively, e.g., via welding. Back sheet 11 is formed from a rip-stop material, e.g., a biocompatible rip-stop nylon. Front sheet 13 may likewise be formed from a rip-stop material, although other materials are also contemplated. Back sheet 11 defines a generally rectangular configuration having a concave or downwardly-bowed upper edge 11 a configured to facilitate position of tracheotomy shield 10 adjacent a patient's neck, as best shown in FIG. 5. Front and back sheet 13, 11, respectively, may either or both be formed from a transparent material. Opening 12 is generally laterally centered on back sheet 11 and is positioned towards the upper end thereof. The configuration of opening 12 will be described in greater detail below.

With continued reference to FIGS. 1-3 and 5, as mentioned above, front sheet 13 is attached to back sheet 11 along at least a portion of the outer peripheries 14a, 14b of back sheet 11 and front sheet 13, respectively, thereby reinforcing the outer peripheries 14a, 14b thereof. More specifically, as best show in FIG. 2, outer periphery 14b of front sheet 13 is welded or otherwise attached to outer periphery 14a of back sheet 11 on three sides, e.g., on the first lateral side, the lower side, and the second lateral side. Front sheet 13 defines a generally U-shaped configuration having a central cut-out 13a. The inner periphery 14c of front sheet 13, which defines cut-out 13a, is free, e.g., unattached, thus defining a three-sided pouch 13b between front sheet 13 and back sheet 11 that is configured to collect secretions and other fluids, inhibiting such secretions and fluids from reaching the tracheotomy wound 23 (FIG. 6), the patient's clothing, or the surrounding area.

Continuing with reference to FIGS. 1-3 and 5, and with additional reference to FIG. 6, opening 12 extends through back sheet 11 and defines a generally circular configuration. More specifically, opening 12 defines a diameter that approximates or is slightly smaller than that of a typical fitting 17 of a tracheotomy tube 19, which defines a diameter of about 15 mm. As a result of this configuration, back sheet 11 is fit about fitting 17 of tracheotomy tube 19, with fitting 17 extending through opening 12, while back sheet 11 establishes a friction-fit, liquid-tight seal about fitting 17 of tracheotomy tube 19. As can be appreciated, the liquid-tight seal inhibits secretions from passing below the back sheet 11 and fitting 17 onto the tracheotomy tube 19, flange 20, dressing 21 and contaminating the tracheotomy wound 23. Further, the friction-fit engagement of back sheet 11 about fitting 17 of tracheotomy tube 19 wholly supports and retains tracheotomy shield 10 on fitting 17 of tracheotomy tube 19, without the need for adhesives, straps, or other supports for retaining tracheotomy shield 10 in place. That is, the configuration of back sheet 11 and opening 12 extending therethrough performs dual functions of establishing a liquid-tight seal about fitting 17 of tracheotomy tube 19 and supporting and retaining tracheotomy shield 10 about fitting 17 of tracheotomy tube 19. The U-shaped configuration of front sheet 13 and the attachment of back and front sheets 11, 13 about their outer peripheries 14a, 14b, respectively, adds structural support and balance such that tracheotomy shield 10 remains in proper position when supported on fitting 17 of tracheotomy tube 19.

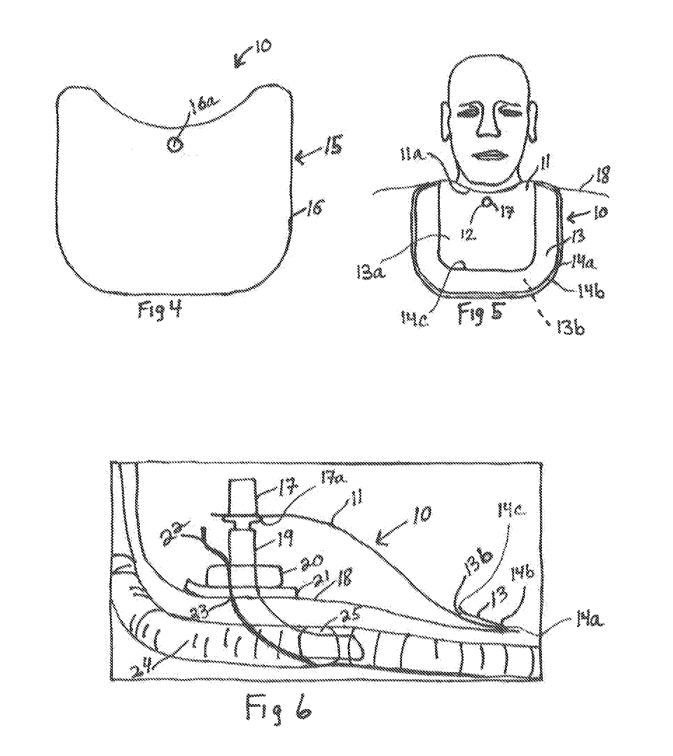

Referring to FIG. 4, another embodiment of a tracheotomy shield is shown generally identified by reference numeral 15. Tracheotomy shield 15 is similar to tracheotomy shield 10 (FIGS. 1-3) except that tracheotomy shield 15 only includes a single sheet 16 defining an opening 16a therethrough. That is, tracheotomy shield 15 does not include a pouch. Tracheotomy shield 15 may otherwise be configured similarly to, and may include any other features of, tracheotomy shield 10 (FIG. 1-3) and, thus, will not be described in further detail herein to avoid unnecessary repetition.

Turning now to FIG. 6, a tracheotomy tube assembly is shown assembled on a patient 18. The tracheotomy tube assembly includes a tracheotomy tube 19 that extends through the tracheotomy wound 23 and into the patient's airway 24. An inflatable cuff 25 creates a seal within the patient's airway about the tube 19. An inflation tube 22 extends along tracheotomy tube 19 and is provided for inflating inflatable cuff 25. An absorbent gauze sponge 21 is positioned on the patient's skin about the tracheotomy wound 23, with a neck flange 20 being positioned atop the absorbent gauze sponge 21. Tracheotomy tube 19 extends from the stoma 23 through an opening in the absorbent gauze sponge 21 and through an opening in the neck flange 20, ultimately coupling to a fitting 17, e.g., a 15 mm fitting. Thus, the patient 18 is able to breathe through the fitting 17 and tube 19.

With continued reference to FIG. 6, and with additional reference to FIG. 5, tracheotomy shield 10, as mentioned above, is fit about fitting 17 to establish a fluid-tight seal thereabout. Fitting 17 further defines and annular shoulder 17a for seating tracheotomy shield 10 thereon and facilitating proper placement of tracheotomy shield 10 about fitting 17. With tracheotomy shield 10 properly positioned about fitting 17, as shown in FIG. 6, any fluid or secretions that are expelled from fitting 17 are shielded from migrating down the tracheotomy tube 19 or onto the patients clothing but, rather, are captured by tracheotomy shield 10 and guided by gravity into pouch 13b.

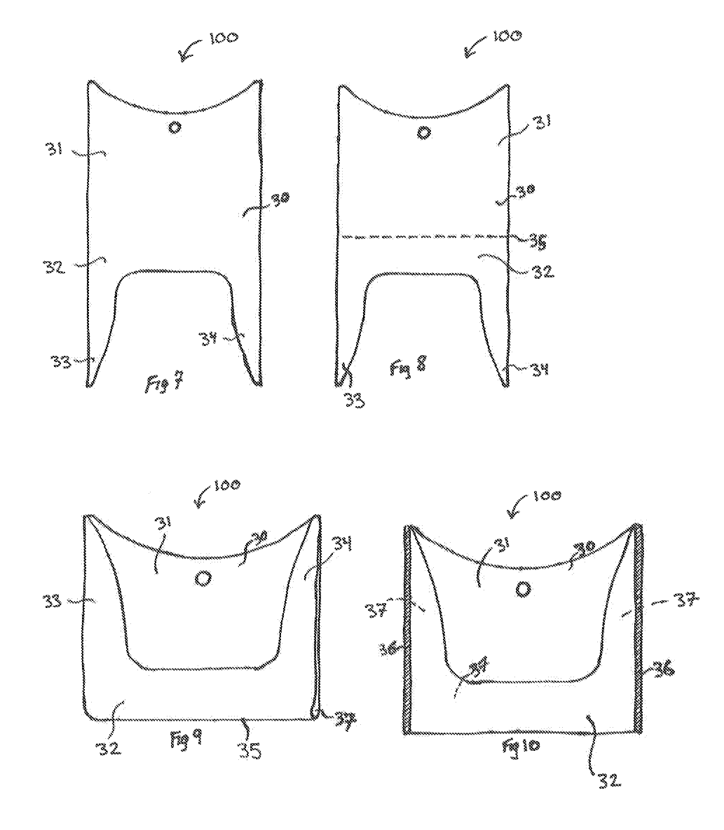

Turning now to FIGS. 7-10, a tracheotomy shield similar to tracheotomy shield 10 (FIGS. 1-6) and including any or all of the features thereof is shown generally identified by reference numeral 100. Tracheotomy shield 100 differs from tracheotomy shield 10 (FIGS. 1-6) mainly in that tracheotomy shield 100, as will be described in greater detail below, is formed from a single sheet of material 30.

Referring to FIGS. 7-8, the single sheet of material 30 is shown including an upper portion 31, and a lower portion 32 defining a pair of spaced-apart legs 33, 34. Similarly as above, tracheotomy shield 100 may be formed from any suitable protective material, e.g., a rip-stop material, and may be transparent. During manufacturing, the single sheet of material 30 is configured to be folded along fold line 35, which is generally disposed between upper and lower portions 31, 32, respectively, of the sheet of material 30.

With reference to FIG. 9, the single sheet of material 30 is shown after folding about fold line 35. More specifically, the single sheet of material 30 is folded such that legs 33, 34 are positioned adjacent the edges of upper portion 31, thus creating a loop or sling-like configuration. As shown in FIG. 10, once folded into position, legs 33, 34 and the corresponding sides of upper portion 31 are joined to one another, e.g., sealed along seal line 36 via welding or other suitable sealing process, to form a pouch area 37 extending along the bottom and sides of tracheotomy shield 100 for catching fluids and secretions.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A tracheotomy shield, comprising:
a first sheet defining an opening extending therethrough, the opening configured to receive a portion of a tracheotomy tube assembly and to establish a liquid-tight seal about the portion of the tracheotomy tube assembly, the first sheet wholly supported by the tracheotomy tube assembly.

2. The tracheotomy shield according to claim 1, wherein the first sheet is formed from a ripstop material.

3. The tracheotomy shield according to claim 1, wherein the first sheet defines a concave upper edge.

4. The tracheotomy shield according to claim 3, wherein the opening is positioned toward the concave upper edge of the first sheet.

5. The tracheotomy shield according to claim 1, further comprising a second sheet disposed atop the first sheet and attached to the first sheet about at least a portion of outer peripheries of the first and second sheets.

6. The tracheotomy shield according to claim 5, wherein the second sheet defines a generally U-shaped configuration and is attached to the first sheet on three sides.

7. The tracheotomy shield according to claim 5, wherein the second sheet defines a free inner peripheral edge forming a pouch between the first and second sheets.

8. The tracheotomy shield according to claim 5, wherein the second sheet is attached to the first sheet via welding.

9. The tracheotomy shield according to claim 5, wherein the first and second sheets are integrally formed as a single sheet.

10. A tracheotomy system, comprising:
a tracheotomy tube configured for positioning within an airway of a patient;
a fitting configured for engagement about a free end of the tracheotomy tube; and
a tracheotomy shield, including:
a first sheet defining an opening extending therethrough, the opening configured to receive a portion of the fitting and to establish a liquid-tight seal about the fitting, the first sheet wholly supported by the fitting.

11. The tracheotomy system according to claim 10, wherein the fitting includes an annular shoulder configured to seat the first sheet thereon.

12. The tracheotomy system according to claim 10, wherein the first sheet is formed from a ripstop material.

13. The tracheotomy system according to claim 10, wherein the first sheet defines a concave upper edge.

14. The tracheotomy system according to claim 10, further comprising a second sheet disposed atop the first sheet and attached to the first sheet about at least a portion of outer peripheries of the first and second sheets.

15. The tracheotomy system according to claim 14, wherein the second sheet defines a generally U-shaped configuration and is attached to the first sheet on three sides.

16. The tracheotomy system according to claim 14, wherein the second sheet defines a free inner peripheral edge forming a pouch between the first and second sheets.

17. The tracheotomy system according to claim 15, wherein the first and second sheets are integrally formed as a single sheet.

18. A tracheotomy shield, comprising:
a first sheet defining an opening extending therethrough, the opening configured to receive a portion of a tracheotomy tube assembly and to establish a liquid-tight seal about the portion of the tracheotomy tube assembly, the first sheet wholly supported by the tracheotomy tube assembly; and
a second sheet defining a generally U-shaped configuration, the second sheet attached to the first sheet about at least a portion of outer peripheries of the first and second sheets to define a pouch therebetween, the second sheet configured to provide structural support and balance to the first sheet to maintain proper positioning of the first sheet when supported by the tracheotomy tube assembly.

19. The tracheotomy shield according to claim 18, wherein at least one of the first and second sheets is formed from a ripstop material.

20. The tracheotomy shield according to claim 18, wherein the first sheet defines a concave upper edge.
